(19) United States
(12) Patent Application Publication
(10) Pub. No.: US 2023/0346217 A1
(43) Pub. Date: Nov. 2, 2023

(54) REDUCING ABBERATIONS IN OPHTHALMIC IMAGING

(71) Applicant: Alcon Inc., Fribourg (CH)

(72) Inventor: Paul R. Hallen, Colleyville, TX (US)

(21) Appl. No.: 18/303,166

(22) Filed: Apr. 19, 2023

Related U.S. Application Data
(60) Provisional application No. 63/363,742, filed on Apr. 28, 2022.

(51) Int. Cl. A61B 3/13

(52) U.S. Cl. CPC A61B 3/13; A61B 3/0025

(57) ABSTRACT

Certain embodiments herein relate to a method for use by an ophthalmic visualization device in providing ophthalmic visualization of an eye of a patient. In certain embodiments, the method includes, receiving an indication indicative of a material occupying a vitreous cavity of the eye. The method further includes determining, based on the indication, adjustment configurations for adjusting one or more optical elements of the ophthalmic visualization device. The method further includes automatically adjusting the one or more optical elements of the ophthalmic visualization device. The method also includes providing visualization of the eye using the adjusted one or more optical elements.

### BACKGROUND

During ophthalmic surgical procedures, such as vitrectomy, surgeons rely on microscopes (e.g., digital microscope), for real time visualization of the optical components of the eye, as well as intraoperative ophthalmic imaging devices, such as optical coherence technology (OCT), to view models of the optical components of the eye. The optical components of the eye include the cornea, aqueous humor, lens, vitreous humor and retina. To provide accurate visualization of the eye and its components and to reconstruct a geometric model of the optical components of the eye, existing microscopes and ophthalmic imaging devices, respectively, rely on the refractive indices of the optical components of the eye, including the vitreous.

Vitrectomy is a type of ophthalmic surgical procedure that treats problems with the retina or the vitreous. A vitrectomy requires the removal of some or all of the vitreous humor (“vitreous”) of the eye. A vitrectomy may be performed as part of a vitreo-retinal procedure for treating conditions such as diabetic traction retinal detachment, diabetic vitreous hemorrhage, macular hole, retinal detachment, epimacular membrane, and many other ophthalmic conditions. Generally, in a vitrectomy, the vitreous is replaced with other material, such as air, expansible gases or other material. As each of these different materials has a different refractive index, however, replacing the vitreous with one of these materials results in a reduction in the visual acuity (e.g., increase in aberrations) with which (1) visualization is provided to the surgeon through a microscope or (2) one or more models of the eye are constructed through an intra-operative ophthalmic imaging device. Such reduction in visual acuity compromises the surgeon's visualization, which in turn impacts the surgeon's clarity and ability to focus, thereby preventing them from determining whether the surgery's goals have been fully achieved. As a result, the surgeon may miss correcting surgical errors that may later need to be corrected through performing additional operations.

### BRIEF SUMMARY

The present disclosure relates generally to methods for adjusting a visualization device or imaging device to reduce aberrations in visualization or modeling of the patient's eye.

Certain embodiments provide a method for use by an ophthalmic visualization device in providing ophthalmic visualization of an eye of a patient. The method includes receiving an indication indicative of a material occupying a vitreous cavity of the eye. The method further includes determining, based on the indication, adjustment configurations for adjusting one or more optical elements of the ophthalmic visualization device. The method further includes automatically adjusting one or more optical elements of the ophthalmic visualization device. The method also includes providing visualization of the eye using the adjusted one or more optical elements.

Certain embodiments provide a method for use by an ophthalmic imaging device in providing ophthalmic imaging of an eye of a patient. The method includes receiving an indication indicative of a material occupying a vitreous cavity of the eye. The method also includes determining, based on the indication, a refractive index of the material. The method also includes generating one or more images or models of the eye based on the refractive index.

The following description and the related drawings set forth in detail certain illustrative features of one or more embodiments.

### BRIEF DESCRIPTION OF THE DRAWINGS

The drawings described herein are for illustrative purposes only, are schematic in nature, and are intended to be exemplary rather than to limit the scope of the disclosure.

FIG. 1 shows a diagram of an example ophthalmic environment including a visualization device, a surgical console, and a patient's eye, according to certain embodiments.

FIG. 2 shows a perspective view of an example the visualization device of FIG. 1, according to certain embodiments.

FIG. 3 shows a diagram illustrative of examples optical elements associated with the visualization device of FIG. 2, according to certain embodiments.

FIG. 4 is a flowchart of a method for adjusting optical components of a visualization device based on an indication indicative of material occupying the patient's vitreous cavity, according to certain embodiments.

FIG. 5 shows a diagram of another example ophthalmic environment including an intra-operative ophthalmic imaging device, a surgical console, and a patient's eye, according to certain embodiments.

FIG. 6 is a flowchart of a method for generating one or more images and/or models, using and intra-operative ophthalmic imaging device, based on an indication indicative of material occupying the patient's vitreous cavity, according to certain embodiments.

The above summary is not intended to represent every possible embodiment or every aspect of the subject disclosure. Rather, the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

While features of the present invention may be discussed relative to certain embodiments and figures below, all embodiments of the present invention can include one or more of the advantageous features discussed herein. In other words, while one or more embodiments may be discussed as having certain advantageous features, one or more of such features may also be used in accordance with various other embodiments discussed herein. In similar fashion, while exemplary embodiments may be discussed below as device, instrument, or method embodiments it should be understood that such exemplary embodiments can be implemented in various devices, instruments, and methods.

During vitrectomy, a surgeon uses a vitrectomy probe to remove the vitreous in order to address diseases and conditions relating to the patient's retina or the vitreous. As the vitreous is removed, the vitreous cavity is infused with an infusion fluid, such as balanced salt solution (BSS), so that the pressure inside the eye is maintained. In certain cases, to address a retinal condition, such as a detached retina, a fluid-air exchange is then performed to replace the infusion fluid with some type of gas, such as air or expansible gases. For example, air may replace the BSS to reattach the retina. In certain other cases, a fluid-liquid perfluorocarbon exchange is performed to replace the infusion fluid that is infused in the eye during removal of the vitreous with liquid perfluorocarbon to attach the retina. In certain other cases, a fluid-silicone oil exchange is performed to replace the infusion fluid that is infused in the eye during removal of the vitreous with silicone oil to attach the retina.

As described above, each one of the materials that may be used to replace the vitreous in the vitreous cavity has a different refractive index. However, when generating imaging data, existing visualization or imaging systems used intra-operatively during a surgical procedure, are not configured to account for the change in the refractive index associated with material exchange that occurs during vitrectomy. In other words, existing visualization or imaging systems may be configured to provide imaging data based on the refractive index of the vitreous. When the vitreous is replaced with other material, the existing visualization or imaging systems' failure to account for the refractive index of the material that has replaced the vitreous, results in aberrations and a deterioration of visual acuity. Note that, in addition to materials that may be used during a vitrectomy to replace the vitreous, in certain cases, the vitreous itself may have degenerated (e.g., floaters may be visible in the vitreous). In general, a degenerative vitreous, across the patient population, may have an average refractive index that is different from an average refractive index of a healthy vitreous across different patients. Therefore, not accounting for the difference between the refractive index of a degenerative vitreous compared to the refractive index of a healthy vitreous may similarly result in aberrations and a deterioration of visual acuity when a surgeon is operating on a patient with a degenerative vitreous.

Accordingly, the embodiments herein describe systems and methods for adjusting a visualization device or imaging device to reduce aberrations in visualization or modeling of the patient's eye.

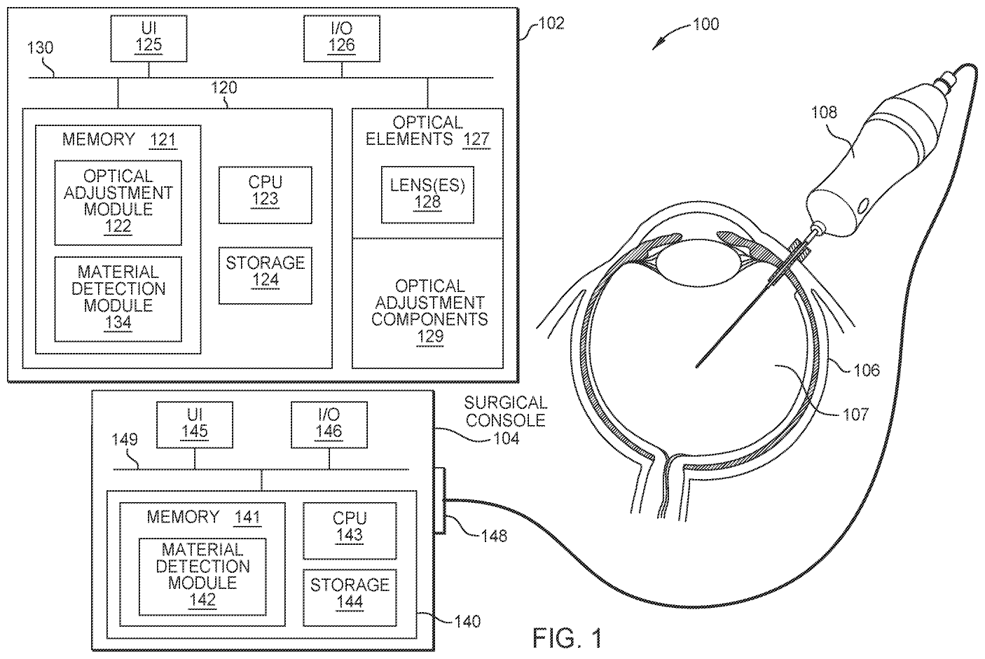

FIG. 1 illustrates a diagram of an example ophthalmic environment 100 including a visualization device 102, a surgical console 104, and a patient's eye 106, according to certain embodiments. For example, ophthalmic environment 100 may correspond to an ophthalmic surgical environment in an operating room where a vitrectomy procedure is being performed. A surgeon may use visualization device 102 to view the patient's eye 106 while performing a vitrectomy using a vitrectomy probe 108 that is powered by surgical console 104.

In certain embodiments, visualization device 102 is a digital microscope, such as three-dimensional stereoscopic digital microscopes (e.g., NGENUITY® 3D Visualization System (Alcon Inc., Switzerland)). As shown, visualization device 102 includes a controller 120, a user interface 125, an interconnect 130, and an I/O (Input/Output) device interface 126, which may allow for the connection of various I/O devices to visualization device 102, optical elements 127, lens(es) 128, as well as optical adjustment components 129.

The controller 120 includes a central processing unit (CPU) 123, a memory 121, and a storage 124. CPU 123 may retrieve and execute programming instructions stored in memory 121. Similarly, CPU 123 may retrieve and store application data residing in memory 121. The interconnect 130 transmits programming instructions and application data, among CPU 123, I/O device interface 126, user interface 125, memory 121, the storage 124, etc. CPU 123 can represent a single CPU, multiple CPUs, a single CPU having multiple processing cores, and the like. Additionally, in certain embodiments, the memory 121 represents volatile memory, such as random access memory (RAM). Furthermore, in certain embodiments, storage 124 may be non-volatile memory, such as a disk drive, solid state drive, or a collection of storage devices distributed across multiple storage systems.

As shown, memory 121 includes an optical adjustment module 122, which refers to an application or set of software instructions for adjusting one or more of optical elements 127 (e.g., lenses 128) based on an indication indicative of the material occupying the vitreous cavity 107 of the patient's eye 106. For example, optical adjustment module 122 may be configured to determine, in real-time or near real-time, the material that occupies vitreous cavity 107 based on the indication. As described above, during a vitreo-retinal procedure, the vitreous is removed and exchanged with other types of material, such as BSS, air, an expansible gas, liquid perfluorocarbon, silicone oil, etc. The material may also include degenerative vitreous. In certain embodiments, the indication may indicate which one of these materials occupies the vitreous cavity. In certain embodiments, the indication may indicate that the material occupying the vitreous cavity includes a combination of vitreous and one of the materials discuss above. In certain embodiments, the indication may indicate a refractive index of the material that occupies the vitreous cavity.

The indication sent to optical adjustment module 122 may be generated in one of a variety of ways. In certain embodiments, the indication may be generated by surgical console 104. For example, surgical console 104 may be configured to determine what operations the surgeon is performing based on user input (e.g., surgeon or assistant) selecting the step of the operation the surgeon is or is planning to engage in. Examples of the steps that may be selected or indicated to surgical console 104 by a user may include the vitrectomy step (e.g., indicating that vitreous is being removed and BSS is being infused), the fluid-air exchange, the fluid-liquid perfluorocarbon exchange, the fluid-silicone oil exchange, etc. A user input indicating the step of the operation can be indicative of the material that is or will shortly (e.g., shortly after the user input is received) be occupying the vitreous cavity.

In certain other embodiments, the indication may be generated by surgical console 104 and/or visualization device 102 using one or more image detection techniques. For example, surgical console 104 and/or visualization device 102 may be configured with image detection software as well as cameras and other necessary components to obtain images of the patient's eye, the surgeon's tools, the surgeon's hands, and other occurrences in the operating room and determine the step of the operations, such as the steps discussed above.

In certain embodiments, the indication may be generated by a material detection module 134 in memory 121. Material detection module 134 may be configured with image detection instructions/software to obtain images generated by one or more optical elements 127 (e.g., including cameras, etc.) and automatically detect the stage of the operation and/or determine the material occupying the vitreous cavity 107. The material may include BSS, air, an expansible gas, liquid perfluorocarbon, silicone oil, degenerative vitreous, etc. Material detection module 134 may also be configured to receive user input from a surgeon indicative of the material occupying the vitreous cavity 107. Based on a determination about the material occupying the vitreous cavity 107, material detection module 134 may then be configured to send an indication to optical adjustment module 122.

Based on an indication, generated through one of the variety of ways described above, optical adjustment module 122 is then configured to cause CPU 123 to send signals through interconnect 130 to optical adjustment components 129 to adjust the position of the one or more of the optical elements 127. For example, optical adjustment components 129 may include electrical, mechanical, and/or electro-mechanical components to adjust the position of one or more lenses 128 of optical elements 127 used to visualize the patient's eye. The signals sent by optical adjustment module 122 may indicate an adjustment criteria for adjusting the one or more optical elements 127. The adjustment criteria is based on the indication indicative of the material occupying the vitreous cavity 107 of the patient's eye 106. For example, the adjustment criteria is based on the refractive index of the material occupying the vitreous cavity. The adjustment criteria may indicate an offset (relative to a starting position) or by how much the position of the one or more optical elements 127 should be adjusted in relation to the patient's eye to account for the change in the refractive index of the material occupying the vitreous cavity 107. Note that the starting position of the one or more optical elements 127 may correspond to a position that allows for the visualization device 102 to provide the best visual acuity or minimum aberrations when viewing the patient's eye prior to vitreous being removed. In other words, the initial or starting position of the one or more optical elements 127 may be configured based on the refractive index of the vitreous.

FIG. 1 also illustrates an example of a surgical console 104. As shown, surgical console 104 includes, without limitation, control module 140, user interface display 145, an interconnect 149, output device 148, and at least one I/O device interface 146, which may allow for the connection of various I/O devices (e.g., keyboards, displays, mouse devices, pen input, etc.) to surgical console 104.

Control module 140 includes a processor or central processing unit (CPU) 143, a memory 141, and storage 144. CPU 143 may retrieve and execute programming instructions stored in the memory 141. Similarly, CPU 143 may retrieve and store application data residing in memory 141. Interconnect 149 transmits programming instructions and application data, among CPU 143, I/O device interface 146, user interface display 145, memory 141, storage 144, output device 148, etc. CPU 143 can represent a single CPU, multiple CPUs, a single CPU having multiple processing cores, and the like. Additionally, memory 141 represents volatile memory, such as random access memory. Furthermore, the storage 144 represents non-volatile memory, such as a disk drive. Although shown as a single unit, storage 144 may be a combination of fixed or removable storage devices, such as fixed disc drives, removable memory cards or optical storage, network attached storage (NAS), or a storage area-network (SAN).

Memory 141 includes instructions, which when executed by the processor, cause surgical console 104 to perform one or more operations. For example, according to embodiments described herein, memory 141 may store a variety of instructions to cause surgical console 104 to operate and/or control the operations of a variety of output devices 148, such as vitrectomy probe 108. In addition, memory 141 also stores a material detection module 142 for determining the type of material occupying the vitreous cavity 107 during one or more steps of a vitreo-retinal procedure and/or generating an indication indicative of the type of material occupying the vitreous cavity 107 during one or more steps of a vitreo-retinal procedure.

For example, material detection module 142 may be configured to determine what operations the surgeon is performing based on user input (e.g., surgeon or assistant) selecting the step of the operation the surgeon is or is planning to engage in. Based on a determination about the operations occurring in real-time, material detection module 142 may be configured to determine the material occupying the vitreous cavity 107 during the determine operation. Material detection module 142 may then send an indication to visualization device 102 indicative of the material occupying the vitreous cavity 107 and/or the refractive index of the material. In certain embodiments, surgical console 104 may further comprise one or more cameras for obtaining images of the patient's eye, surgeon's tools/hands, etc. In such embodiments, material detection module 142 may be configured with image detection instructions to obtain the images generated by the cameras and/or automatically detect the stage of the operation and determine the material occupying the vitreous cavity 107. Based on such a determination, an indication is sent by material detection module 142 to visualization device 102 indicative of the material occupying the vitreous cavity 107 and/or the refractive index of the material.

As shown, surgical console 104 also includes output devices 148, which may refer to a variety of probes (e.g., surgical probes, illumination probes, laser probes, and/or other probes and devices typically used for ophthalmic surgical procedures). One of output devices 148 is shown as vitrectomy probe 108, which is used to remove the vitreous from the vitreous cavity 107. In certain embodiments, material detection module 142 may be configured to determine the material occupying the vitreous cavity based on what probe is being used. For example, material detection module 142 may receive a signal that vitrectomy probe 108 is being used, indicating that the vitreous is being removed. Material detection module 142 may also receive a signal indicating that an infusion probe for infusing air or BSS or other types of material is being used. Based on these signals, material detection module 142 may be configured to determine or, at least, estimate, what material is occupying the vitreous cavity in real-time or near real-time.

### Example Visualization Device

Visualization device 102 may be or include one of a variety of imaging and/or microscopic devices.

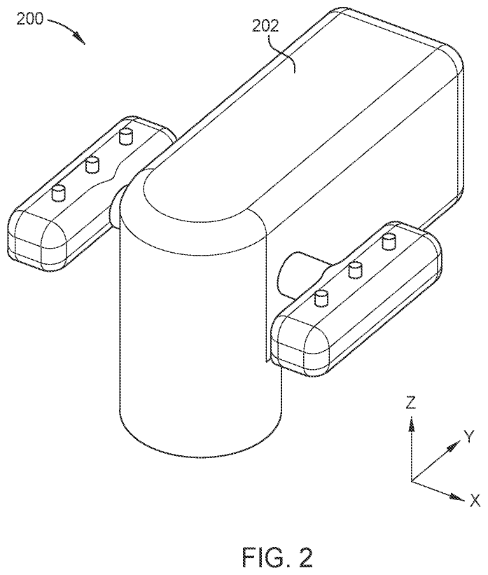

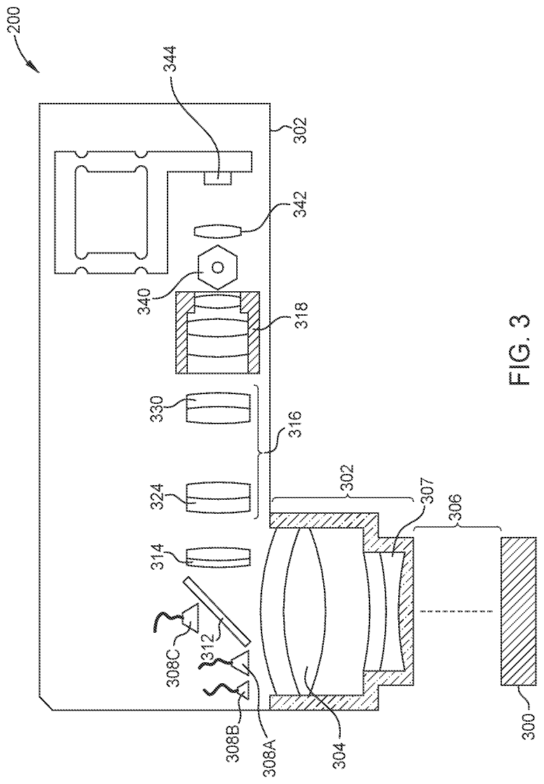

FIG. 2 illustrates a perspective view of a stereoscopic visualization camera 200, which may be an example of visualization device 102 of FIG. 1. As shown in FIG. 2, the stereoscopic visualization camera 200 includes a housing 202 configured to enclose optical elements, lens motors (e.g., actuators), and signal processing circuity. FIG. 3 shows an example arrangement and positioning of the optical elements of the stereoscopic visualization camera 200. In some cases, the arrangement and positioning of the optical elements of the stereoscopic visualization camera 200 forms two parallel optical paths to generate a left view and a right view. The parallel optical paths correspond to a human's visual system such that the left view and right view, as displayed on a stereoscopic display, appear to be separated by a distance that creates a convergence angle of, for example, roughly 6 degrees, which is comparable to the convergence angle for an adult human's eyes viewing an object at approximately 4 feet away, thereby resulting in stereopsis. In some embodiments, image data generated from the left view and right view are combined together on the display monitor(s) to generate a stereoscopic image of a target surgical site or scene.

A stereoscopic view, as compared to a monoscopic view, mimics the human visual system much more closely. A stereoscopic view provides depth perception, distance perception, and relative size perception to provide a realistic view of a target surgical site to a surgeon. For procedures such as retinal surgery, stereoscopic views are useful because surgical movements and forces are so small that the surgeon cannot feel them. Providing a stereoscopic view helps a surgeon's brain magnify tactile feel when the brain senses even minor movements while perceiving depth.

FIG. 3 shows a side view of the example stereoscopic visualization camera 200 with the housing 202 being transparent to expose the optical elements. The optical elements shown in FIG. 3 may be part of a left optical path and may generate the left view. It should be appreciated that the arrangement and positioning of optical elements in a right optical path in stereoscopic visualization camera 200 (e.g., generating the right view) may generally be identical to the left optical path.

The example stereoscopic visualization camera 200 is configured to acquire images of a target surgical site 300 (also referred to as a scene or field-of-view) at a working distance 306 above the target surgical site 300. The target surgical site 300 includes the patient's eye. Images from the target surgical site 300 are received at the stereoscopic visualization camera 200 via a main objective assembly 302, which includes the front working distance lens 307 and a rear working distance lens 304.

To illuminate the target surgical site 300, the example stereoscopic visualization camera 200 includes one or more lighting sources, such as a near-infrared (“NIR”) light source 308B, and a near-ultraviolet (“NUV”) light source 308C. In other examples, the stereoscopic visualization camera 200 may include additional or fewer (or no) light sources. For instance, the NIR and NUV light sources may be omitted. The example light sources 308 are configured to generate light, which is projected to the target surgical site 300. The generated light interacts and reflects off the target scene, with some of the light being reflected to the main objective assembly 302. Other examples may include external light sources or ambient light from the environment.

The projection of the light from light sources 308 through the main objective assembly provides the benefit of changing the lighted field-of-view based on the working distance 306 and/or focal plane. Since the light passes through the main objective assembly 302, the angle at which light is projected changes based on the working distance 306 and corresponds to the angular field-of-view. This configuration accordingly ensures the field-of-view is properly illuminated by the light sources 308, regardless of working distance or magnification.

Further, as illustrated in FIG. 3, the stereoscopic visualization camera 200 includes a deflecting element 312. In some cases, the deflecting element 312 may be configured to transmit a certain wavelength of light from the NUV light source 308C to the target surgical site 300 through the main objective assembly 302. The deflecting element 312 may also be configured to reflect light received from the target surgical site 300 to downstream optical elements, including a front lens set 314 for zooming and recording. In some embodiments, the deflecting element 312 may filter light received from the target surgical site 300 through the main objective assembly 302 so that light of certain wavelengths reaches the front lens set 314.

The deflecting element 312 may include any type of mirror or lens to reflect light in a specified direction. In an example, the deflecting element 312 includes a dichroic mirror or filter, which has different reflection and transmission characteristics at different wavelengths. The stereoscopic visualization camera 200 of FIG. 3 includes a single deflecting element 312, which provides light for both the right and left optical paths. In other examples, the stereoscopic visualization camera 200 may include separate deflecting elements for each of the right and left optical paths. Further, a separate deflecting element may be provided for the NUV light source 308C.

The example stereoscopic visualization camera 200 of FIG. 3 includes one or more zoom lenses to change a focal length and angle of view of the target surgical site 300 to provide zoom magnification. In the illustrated example of FIG. 3, the zoom lenses include the front lens set 314, a zoom lens assembly 316, and a lens barrel set 318. In some cases, the zoom lenses may include additional lens(es) to provide further magnification and/or image resolution. Note that one or more optical adjustment components (e.g., optical adjustment components 129) may be used (not shown) to adjust the position and/or angle of one or more of the zoom lenses, including front lens set 314, a zoom lens assembly 316, and/or a lens barrel set 318, and/or one or more of the lenses in main objective assembly 302, including front working distance lens 307 and a rear working distance lens 304. As described above, the optical adjustment components may include mechanical, electrical, and/or electromechanical components for adjusting lenses in optical devices, as known to one of ordinary skill in the art.

The front lens set 314 includes a right front lens for the right optical path and a left front lens for the left optical path. The lenses left and right front lenses may each include a positive converging lens to direct light from the deflecting element 312 to respective lenses in the zoom lens assembly 316. A lateral position of the left and right front lenses accordingly defines a beam from the main objective assembly 302 and the deflecting element 312 that is propagated to the zoom lens assembly 316.

The example zoom lens assembly 316 forms a focal zoom system for changing the size of a field-of-view (e.g., a linear field-of-view) by changing a size of the light beam propagated to the lens barrel set 318. The zoom lens assembly 316 includes a front zoom lens set 324 with a right front zoom lens and a left front zoom lens. The zoom lens assembly 316 also includes a rear zoom lens set 330 with a right rear zoom lens and a left rear zoom lens.

The size of a light beam for each of the left and right optical paths is determined based on a distance between the front zoom lenses in the front zoom lens set 324, the rear zoom lenses in the rear zoom lens set 330, and the lens barrel set 318. Generally, the size of the optical paths reduces as the rear zoom lenses in the rear zoom lens set 330 move toward the lens barrel set 318 (along the respective optical paths), thereby decreasing magnification. In addition, the front zoom lenses in the front zoom lens set 324 may also move toward (or away from) the lens barrel set 318 (such as in a parabolic arc), as the rear zoom lenses in the rear zoom lens set 330 move toward the lens barrel set 318, to maintain the location of the focal plane on the target surgical site 300, thereby maintaining focus.

The front zoom lenses in the front zoom lens set 324 may be included within a first carrier while the rear zoom lenses in the rear zoom lens set 330 are included within a second carrier. Each of the carriers may be moved on tracks (or rails) along the optical paths such left and right magnification may be uniformly adjusted (e.g., increased or decreased). Altogether, the front lens set 314, the zoom lens assembly 316, and the lens barrel set 318 are configured to achieve an optical zoom, such as between 5× to about 20×, such as at a zoom level that has diffraction-limited resolution.

After the light from the target surgical site 300, the light in each of the right and left optical paths may pass through one or more optical filters 340 (or filter assemblies) to selectively transmit desired wavelengths of light. The light in each of the right and left optical paths may then pass through a final optical element set 342 that is configured to focus light received from the optical filter 340 onto the optical image sensor 344.

As shown, the stereoscopic visualization camera 200 of FIG. 3 includes the optical image sensor 344, which may be configured to acquire and/or record incident light that is received from the final optical element set 342. The optical image sensor 344 includes a right optical image sensor configured to record light propagating along the right optical path and generate right image data associated with the right optical path. Additionally, the optical image sensor 344 also includes a left optical image sensor configured to record light propagating along the left optical path and generate left image data associated with the left optical path. After the right and left image data are created, one or more processors may synchronize and combine the left and right image data to generate a stereoscopic image. Additionally, the one or more processors may be configured to convert a plurality of stereoscopic images into stereoscopic video data for display to a user of the stereoscopic visualization camera 200 on a display monitor, such as a stereoscopic display.

Additional aspects of the stereoscopic visualization camera 200 may be found in U.S. Pat. No. 11,058,513, titled “STEREOSCOPIC VISUALIZATION CAMERA AND PLATFORM,” the entirety of which is incorporated herein by reference.

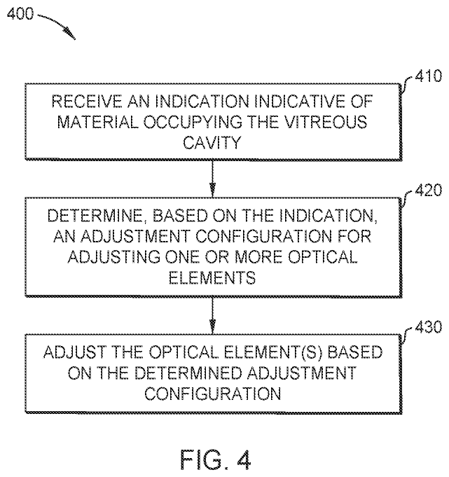

FIG. 4 illustrates the method for adjusting one or more optical elements of a visualization device, such as visualization device 102, based on an indication of a material occupying the vitreous cavity, in accordance with certain embodiments.

At 410, the visualization device receives an indication indicative of material occupying the vitreous cavity. In certain embodiments, receiving the indication can include the visualization device receiving the indication from an entity other than the visualization device itself (e.g., a surgical console). In certain other embodiments, receiving the indication can include an optical adjustment module (e.g., optical adjustment module 122) of the visualization device receiving the indication from a material detection module (e.g., material detection module 134) of the visualization device.

At 420, the visualization device determines, based on the indication, an adjustment configuration for adjusting the one or more optical elements.

At 430, the visualization device adjusts (e.g., automatically) the one or more optical elements based on the determined adjustment configuration. After the visualization device adjusts the one or more optical elements, the visualization device provides visualization of the eye based on the adjusted one or more optical elements, for example, with reduced aberrations.

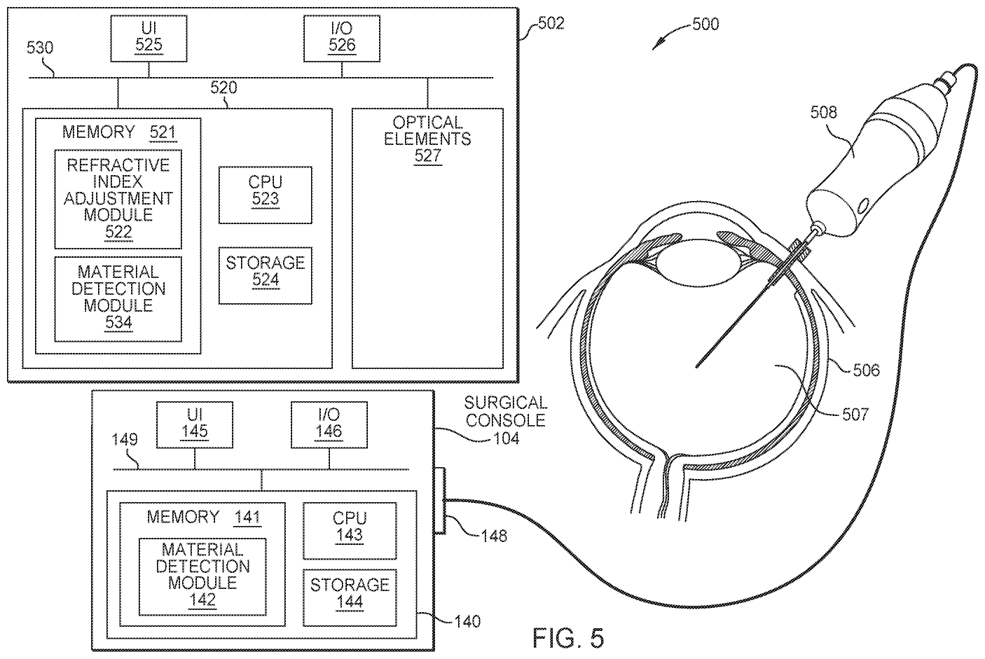

FIG. 5 illustrates a diagram of another example ophthalmic environment 500 including an imaging device 502, a surgical console 104, and a patient's eye 506, according to certain embodiments. For example, the operating environment may correspond to an ophthalmic surgical environment in an operating room where a vitrectomy procedure is being performed. A surgeon may use the imaging device 502 to generate models and/or images of the patient's eye while performing a vitrectomy using a vitrectomy probe 508 that is powered by surgical console 104.

As shown, imaging device 502 includes a controller 520, a user interface 525, an interconnect 530, and an I/O device interface 526 which may allow for the connection of various I/O devices to imaging device 502. The controller 520 includes a central processing unit (CPU) 523, a memory 521, and a storage 524. CPU 523 may retrieve and execute programming instructions stored in memory 521. Similarly, CPU 523 may retrieve and store application data residing in memory 521. The interconnect 530 transmits programming instructions and application data, among CPU 523, I/O device interface 526, user interface 525, memory 521, storage 524, etc. CPU 523 can represent a single CPU, multiple CPUs, a single CPU having multiple processing cores, and the like. Additionally, in certain embodiments, the memory 521 represents volatile memory, such as random access memory (RAM). Furthermore, in certain embodiments, storage 524 may be non-volatile memory, such as a disk drive, solid state drive, or a collection of storage devices distributed across multiple storage systems.

As shown, memory 521 includes a refractive index adjustment module 522, which refers to an application or set of software instructions for adjusting one or more refractive indices used to generate a model or image based on an indication indicative of the material occupying the vitreous cavity 507 of the patient's eye 506. For example, refractive index adjustment module 522 may be configured to determine, in real-time or near real-time, the material that occupies vitreous cavity 507 based on the indication and adjust the refractive index used for the vitreous in formulas used for generating imaging data associated with the eye. Such formulas and software instructions for generating imaging data are also stored in storage 524 and memory 521. Adjusting the refractive index used for the vitreous in such formulas includes changing the refractive index of vitreous to the refractive index of the material that occupies the vitreous cavity in real-time. As described above, during a vitreo-retinal procedure, the vitreous is removed and exchanged with other types of material, such as BSS, air, an expansible gas, liquid perfluorocarbon, silicone oil, etc. Also, in certain cases, the material may include degenerative vitreous. In certain embodiments, the indication may indicate which one of these materials occupies the vitreous cavity. In certain embodiments, the indication may indicate that the material occupying the vitreous cavity includes a combination of vitreous and one of the materials discuss above. In certain embodiments, the indication may indicate a refractive index of the material that occupies the vitreous cavity.

In some embodiments, the imaging device 502 comprises an OCT device. Generally, in an OCT device, imaging data (single or multi-dimensional images or models) is generated by directing a signal (e.g., incident beam or waves) into the eye and obtaining and analyzing the back reflected waves, which reveal information about various optical components of the eye. The analysis of the back reflected waves in an OCT device is typically performed using estimated refractive indices of the various optical components of the eye. The optical components of a patient's eye include the cornea, retina, lens, aqueous humor and vitreous.

However, when one of these optical components, such as the vitreous, is removed and replaced with another material, in an existing OCT device, the surgeon may experience an increase in aberrations. This increase in aberrations is the result of using the refractive index of the vitreous when generating imaging data, while the vitreous has been removed and replaced with another material. Therefore, certain embodiments described herein relate to configuring an OCT device to change the refractive index of the vitreous, to the refractive index of the material that has replaced vitreous, in formulas used for generating imaging data based on an indication about the material. In certain embodiments, refractive index adjustment module 522 is configured with refractive indices of the various materials such as BSS, air, an expansible gas, liquid perfluorocarbon, silicone oil, degenerative vitreous, etc. Therefore, when, for example, imaging device 502 receives an indication that air is currently occupying the vitreous cavity, the refractive index of the air may be substituted in the formulas used by the imaging device 502 to ensure accurate models and/or images are produced.

The indication of the material occupying the vitreous cavity, sent to refractive index adjustment module 522, may be generated in one of a variety of ways. In certain embodiments, the indication may be generated by surgical console 104, as described previously in relation to FIG. 1. In certain other embodiments, the indication may be generated by surgical console 104 and/or imaging device 502 using one or more image detection techniques, as described previously in relation to FIG. 1. In yet certain other embodiments, the indication may be generated by a material detection module 534 in memory 521, as described previously in relation to FIG. 1.

Based on an indication generated through one of the variety of ways described above, refractive index adjustment module 522 is then configured to cause CPU 523 to adjust the initial refractive index (e.g., refractive index of the vitreous) used in the various formulas to generate models and/or images of the eye. For example, refractive index adjustment module 522 may provide an updated refractive index value corresponding to the material occupying the eye for use by an imaging module of imaging device 502 in executing various formulas to model the eye. The imaging module refers to a set of instructions that may execute in memory 521 to generate imaging data and/or models of the eye using various formulas or algorithms, as may be known to one of ordinary skill in the art. Note that although an OCT device was used in the description above as an example of imaging device 502, in certain other embodiments, imaging device 502 may be one of a fundus photography device, an ultrasound device, a scanning laser ophthalmoscopy (SLO) device, and/or any other type of diagnostic or imaging device that relies on the refractive index of the various components of the eye, including the vitreous, for generating imaging data and/or models of the eye.

FIG. 5 also illustrates an example of a surgical console 104, previously described in relation to FIG. 1. A description relating to the components of surgical console 104 was provided in more detail previously and, therefore, is omitted here for brevity. As discussed, material detection module 142 is configured to make a determination about the material occupying the vitreous cavity and may transmit an indication indicative of such material to refractive index adjustment module 522 of imaging device 502.

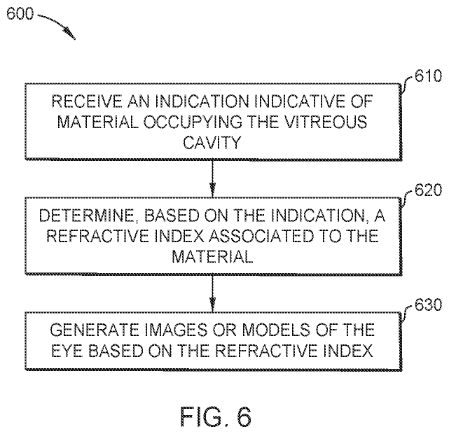

FIG. 6 illustrates the method for generating one or more images and/or models, using and intra-operative ophthalmic imaging device, such as imaging device 502, based on an indication indicative of material occupying the patient's vitreous cavity, according to certain embodiments.

At 610, the imaging device receives an indication indicative of material occupying the vitreous cavity. In certain embodiments, receiving the indication can include the imaging device receiving the indication from an entity other than the imaging device itself (e.g., a surgical console). In certain other embodiments, receiving the indication can include the refractive index adjustment module (e.g., refractive index adjustment module 522) of the visualization device receiving the indication from a material detection module (e.g., material detection module 534) of the visualization device.

At 620, the imaging device determines, based on the indication, a refractive index associated with the material.

At 630, the imaging device generates one or more images and/or models of the patient's eye based on the refractive index associated with the material occupying the patient's vitreous cavity. For example, the imaging device may be configured to automatically reconfigure an imaging module (e.g., stored in memory 521) or the settings of the imaging device (e.g., imaging device 502) with the refractive index associated with the material occupying the patient's vitreous cavity. As a result, the imaging module or settings may use the updated refractive index in formulas and/or algorithms used for generating imaging data and/or models of the eye.

### EXAMPLE EMBODIMENTS

Embodiment 1: A method for use by an ophthalmic imaging device in providing ophthalmic imaging of an eye of a patient, comprising: receiving an indication indicative of a material occupying a vitreous cavity of the eye; determining, based on the indication, a refractive index of the material; and generating one or more images or models of the eye based on the refractive index.

Embodiment 2: The method of embodiment 1, wherein the indication is received from a surgical console based on a determination made by the surgical console about the material occupying the vitreous cavity.

Embodiment 3: The method of embodiment 2, wherein the determination is made by the surgical console based on performing image detection techniques on images of an operation being performed on vitreous inside the vitreous cavity.

Embodiment 4: The method of embodiment 2, wherein the determination is made by the surgical console based on a type of one or more probes being used during an operation being performed using the surgical console.

Embodiment 5: The method of embodiment 1, wherein the indication is based on a determination made by the ophthalmic imaging device about the material occupying the vitreous cavity.

Embodiment 6: The method of embodiment 1, wherein the generating comprises automatically reconfiguring an imaging module of the ophthalmic imaging device with the refractive index, and wherein the generating is performed by the imaging module.

What is claimed is:

1. A method for use by an ophthalmic visualization device in providing ophthalmic visualization of an eye of a patient, comprising:
receiving an indication indicative of a material occupying a vitreous cavity of the eye;
determining, based on the indication, adjustment configurations for adjusting one or more optical elements of the ophthalmic visualization device;
automatically adjusting the one or more optical elements of the ophthalmic visualization device; and
providing visualization of the eye using the adjusted one or more optical elements.

2. The method of claim 1, wherein the indication is received from a surgical console based on a determination made by the surgical console about the material occupying the vitreous cavity.

3. The method of claim 2, wherein the determination is made by the surgical console based on user input provided to the surgical console.

4. The method of claim 3, wherein the user input indicates a step of an operation being performed or that is about to be performed using the surgical console.

5. The method of claim 2, wherein the determination is made by the surgical console based on performing image detection techniques on images of an operation being performed on vitreous inside the vitreous cavity.

6. The method of claim 2, wherein the determination is made by the surgical console based on a type of one or more probes being used during an operation being performed using the surgical console.

7. The method of claim 1, wherein the indication is based on a determination made by the ophthalmic visualization device about the material occupying the vitreous cavity.

8. The method of claim 1, wherein the adjustment configurations indicate an offset relative to a starting position of the one or more optical elements, and wherein the one or more optical elements are automatically adjusted based on the offset.

9. The method of claim 1, wherein the one or more optical elements comprise one or more lenses.

10. The method of claim 1, wherein the ophthalmic visualization device is a digital microscope.

11. The method of claim 1, wherein the ophthalmic visualization device is a stereoscopic visualization camera.

12. A method for use by an ophthalmic imaging device in providing ophthalmic imaging of an eye of a patient, comprising:
receiving an indication indicative of a material occupying a vitreous cavity of the eye;
determining, based on the indication, a refractive index of the material; and
generating one or more images or models of the eye based on the refractive index.

13. The method of claim 12, wherein the indication is received from a surgical console based on a determination made by the surgical console about the material occupying the vitreous cavity.

14. The method of claim 13, wherein the determination is made by the surgical console based on user input provided to the surgical console.

15. The method of claim 14, wherein the user input indicates a step of an operation being performed or that is about to be performed using the surgical console.
